(19) United States
(12) Patent Application Publication
(10) Pub. No.: US 2009/0132046 A1
(43) Pub. Date: May 21, 2009

(54) Implant for Use with an Osteotomy Plate

(75) Inventor: Gregoire Larche, Cholet (FR)

Correspondence Address: MILES & STOCKBRIDGE PC, 1751 PINNACLE DRIVE, SUITE 500, MCLEAN, VA, 22102-3833, US

(73) Assignee: D.L.P., La Haye Fouassiere (FR)

(21) Appl. No.: 11/920,348

(22) PCT Filed: May 18, 2006
(86) PCT No.: PCT/FR2006/001127

§ 371 Date: Jan. 13, 2009

(30) Foreign Application Priority Data
May 20, 2005 (FR) 0505084

(51) Int. Cl. A61F 2/08

(52) U.S. Cl. 623/14.12

(57) ABSTRACT

The subject of the invention is an implant made of bioinert material to be used with an osteotomy plate to correct the alignment of the lower limbs by addition, this implant having a housing that opens onto its upper and lower surfaces, this implant being characterized in that it has sufficient mechanical strength to transmit and/or absorb the loads to which it is subjected so as to protect a bone precursor placed in the housing and is extractible without notably weakening the bone reconstruction, and to this end the housing that houses the bone precursor has a hollow shape and the implant occupies a volume on the order of 10 to 35% of the osteotomy site.

The invention relates to an implant to be used with an osteotomy plate to correct the alignment of the lower limbs by addition.

More particularly, but not by way of limitation, it is intended for tibial osteotomy for valgization.

The lower limb is composed of the femur, the tibia/fibula and the foot.

In the majority of subjects, there is an alignment of the femur and the tibia that allows the knee joint to work normally and thus to transfer loads.

However, certain subjects may be diagnosed with genu valgum or genu varum, which causes uneven wear on the cartilage of the tibial plateau. This problem is accentuated by the fact that the loads are not evenly distributed across the entire tibial plateau.

In fact, it is known that 70% of loads pass through the inner part of the tibial plateau, and thus 30% pass through the outer part.

There is also a distribution between the front and the rear, which is on the order of 60% on the front and 40% on the posterior part.

As a result of this particular distribution and other problems such as excess weight, abnormal wear can appear rapidly when the patient suffers from a genu varum.

It is therefore advisable to operate before it becomes necessary to install a total knee prosthesis.

This operation consists of realigning the tibial with the femur by internal addition.

For valgization of the tibia, one proceeds as follows:

An osteotomy line is cut into the inner metaphyseal zone.

This cut must be precise and must preserve a part of the bone which will serve as an external hinge (osteoclasis).

Using a wedge inserted into the cut line, the axis of the tibia is angularly corrected relative to the femur in order to realign it.

This correction must then be immobilized by an appropriate means during the time it takes for the bone to regenerate and consolidate.

An osteotomy plate attached to the inner surface of the tibia is generally used.

This plate must absorb all of the mechanical loads during the time it takes for the bone to form and fill in the space created.

It generally takes six to nine months for the bone to grow back.

In the corrected position, the two opposing surfaces formed by the cut are separated and delimit a wedge-shaped volume.

There a wedge known from US 2003/0105526 that extends across nearly the entire surface of the cut and is intended to remain in place permanently. Only the cortical bone will regenerate if the wedge is made of stainless steel; thus, an open-cell material is used so that the bone will grow back everywhere.

This wedge is not meant to be removed.

Since this operation is often used to delay the insertion of a knee prosthesis, in order for this wedge not to impede the implantation of a knee prosthesis, the wedge is horseshoe-shaped so that a prosthesis shaft may subsequently be inserted into the hollow of the wedge, which is not extractible.

Independently from this wedge, in order for the wedge-shaped space created after resection to fill in faster, it is filled with natural bone, for example harvested from the iliac crest, or a bone substitute which will specifically act as a catalyst to accelerate the formation of the bone.

The upper and lower surfaces of this bone precursor are in contact with the surfaces of the bone that must be rejoined.

It is generally preferable to use a bone substitute because it avoids the need for the surgery required to harvest natural bone. This bone substitute will have a shape that is adapted so as to nearly fill the space created during the osteotomy. Thus, this bone substitute covers both the cortical and spongy bone.

There are two types of bone substitutes: one is highly friable while the other is stronger.

Some surgeons work exclusively with the dense substitute, others with the friable substitute.

The dense, and therefore stronger, bone substitute is resorbed far less rapidly than the friable substitute; however, the dense bone substitute is not designed to absorb substantial compression loads.

Even when the dense substitute is used, the load on the tibia must be reintroduced very gradually, since bone production takes a relatively long time due to the fact that the pores of this dense substitute are colonized far less rapidly.

Unfortunately, it has been observed that some patients do not follow the post-operative protocol, which causes problems in terms of bone formation.

In essence, the osteotomy plate is designed to withstand and transmit loads, but like any metallic material, it has a certain pliability, and consequently the loads on the osteosynthesis plate generate micro-movements which induce shear forces between the surfaces of the bone and those of the substitute.

Thus, there is no normal osteoconduction.

In that case, encapsulation and fibrosis is observed, resulting in pseudoarthritis.

The operation must then be redone.

Surgeons are therefore uncomfortable with a bone substitute, particularly when it is dense.

Thus, instead of using an osteotomy plate, a wedge-shaped implant known from US 2003/0105526 is used, the size of which substantially corresponds to the cuneiform volume and which is inserted into the open space during the valgization. This wedge is held in place by screws which pass through this implant and are screwed into the bone.

This implant can be made of stainless steel, titanium, ceramic, etc.

The bone will form around this implant, which occupies a large part of the cuneiform volume.

This implant will absorb all loads. It is not necessary to insert an osteotomy plate.

Because of the absence of an osteotomy plate, the reintroduction of loads onto the tibia is very long, and the bone that grows will constantly be subject to microtraumas. Thus, the same problems exist as in the case of the osteotomy plate.

As mentioned above, the hollow is intended to be able to accommodate a prosthesis shaft. It is not intended to house a bone precursor.

There is also a hydroxyapatite wedge, known from U.S. Pat. No. 5,766,251, that is pierced in its center with a channel for the insertion of a bone precursor.

The bone begins to grow through the channel, after which this wedge is gradually colonized by the bone and cannot be removed. Moreover, the bone grows back through this channel, so even if one wished to extract this wedge, it would be impossible.

The presence of this wedge presents a problem when inserting a prosthesis because the material composing this wedge does not have the same properties as bone.

There is another vertebral implant known from US2003/0125739 which, once in place, cannot be removed.

The invention proposes to provide a solution to the various problems mentioned above.

To this end, the subject of the invention is a bioinert implant to be used with an osteotomy plate to correct the alignment of the lower limbs by addition, this implant having a housing that opens onto its upper and lower surfaces, this implant being characterized in that it has sufficient mechanical strength to transmit and/or absorb the loads to which it is subjected so as to protect the bone precursor placed inside the housing and is extractible without any notable weakening of the bone reconstruction, and to this end the housing that houses the bone precursor has a hollow shape and the implant occupies a volume on the order of 10 to 35% of the osteotomy site.

The invention will be clearly understood with the aid of the following description, given as a nonlimiting example in reference to the drawings, which schematically represent:

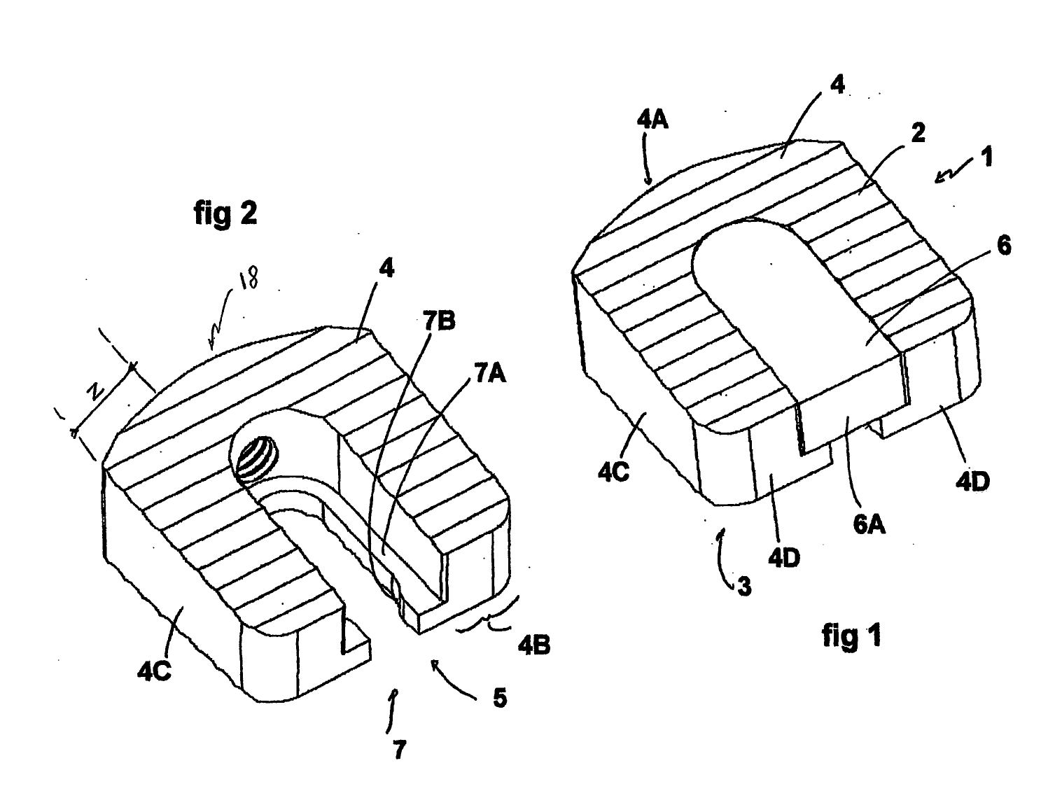

FIG. 1: an implant

FIG. 2: the implant without the inner bone substitute

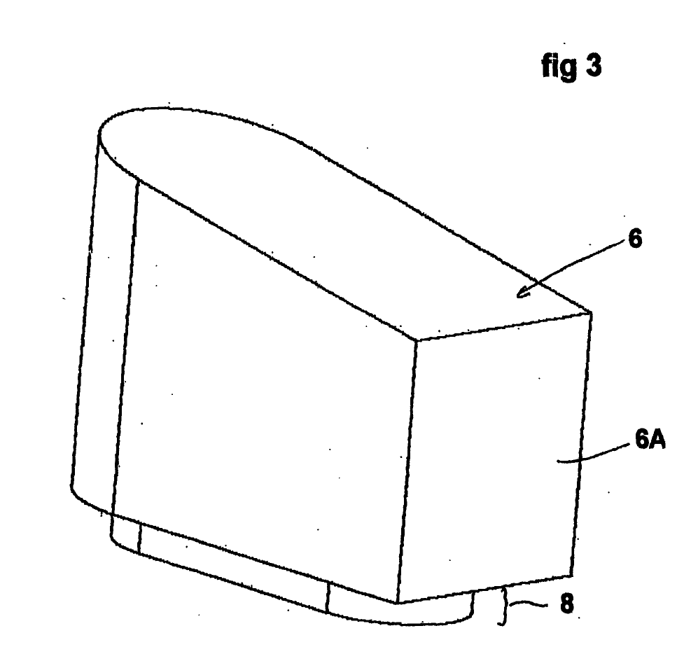

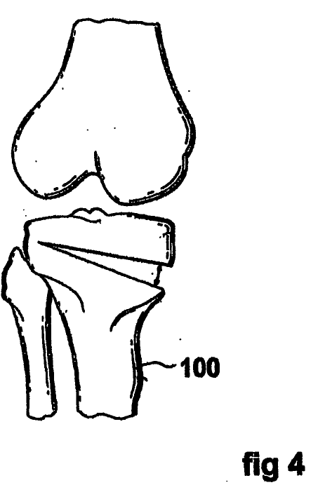

FIG. 3: the inner bone substitute to be attached to the part of the implant shown in FIG. 2

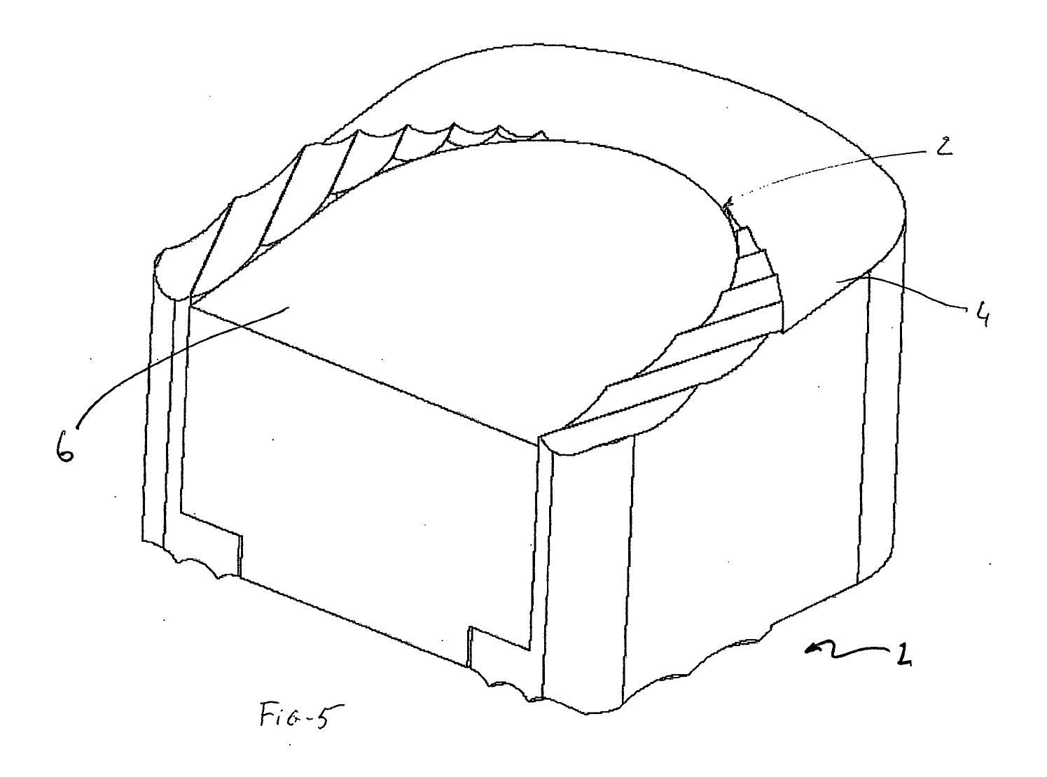

FIG. 4: a tibia after osteotomy and before the insertion of the implant and the osteosynthesis plate

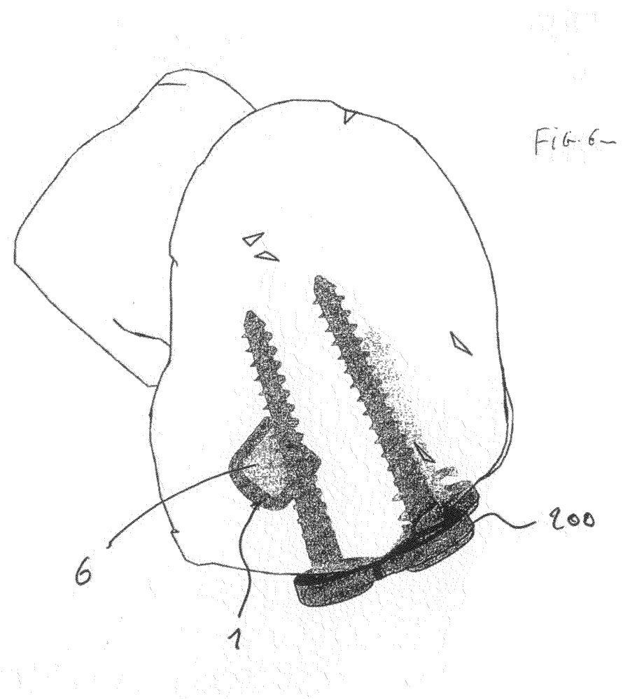

FIG. 5: a variant of an implant

FIG. 6: a see-through illustration of the installed implant and osteotomy plate

Referring to the drawings, we see that in order to correct the axis of the lower limbs, an osteotomy for valgization by addition is performed.

FIG. 4 represents a tibial osteotomy for valgization.

After the osteotomy is performed, the tibia 100 is realigned by means of a wedge (not shown) adapted to the angle chosen for the correction.

The angle formed after correction may be clearly seen.

The alignment of the limb is maintained by an osteotomy plate 200 attached to the anterointernal surface with screws; then, after the wedge is removed, the implant 1 is inserted.

This implant is made of bioinert material and may include a bone regrowth activator.

This implant is temporary, i.e., it is removed after the bone has reformed.

It is made of bioinert material so that it will not be colonized.

The volume of this implant is much smaller than the volume of the osteotomy site. It is on the order of 10 to 35% of the volume of the osteotomy site, the upper and lower surfaces of the implant being in contact with the bone.

Preferably, this volume is on the order of 10 to 20% of the volume of the osteotomy site. The surface area occupied is very small. For example, the surface area occupied is generally on the order of two to three square centimeters.

It may be clearly seen in FIG. 6 that the size of this implant is small.

The length of the implant is shorter than the radius of the cross-section of the bone, measured at the level of the cut. It is generally placed at the level of the inner lateral ligament, the rear edge being aligned with the cortex. In a frontal plane, the implant never extends past the plane passing through the internal mass of the tibial spines.

The thickness of the implant 1 decreases from its outer wall in order to fit into the cuneiform space created by the osteotomy and the angular change of the tibia.

The upper 2 and lower 3 surfaces of the implant 1 determine the secant planes whose angle substantially corresponds to the desired correction so that the opposing sides of the osteotomy site rest against the upper and lower surfaces of the implant.

The implant has a housing A that opens onto its upper and lower surfaces, and sufficient mechanical strength to transmit and/or absorb the loads to which it is subjected.

The mechanical strength of this implant combines with the mechanical strength of the osteotomy plate to limit micro-movements.

As long as it is in place, the implant protects the bone precursor.

Preferably, its mechanical properties will be similar to those of the cortex of the bone.

For example, it could be a polymer, possibly reinforced with carbon fibers, which has the advantage of being X-ray transparent.

For example, it could be polyetheretherketone, known by the acronym “PEEK.”

Advantageously, the housing A receives a bone regrowth activator or bone precursor such as harvested bone or a bone substitute. This regrowth activator occupies approximately 70% of the total volume of the implant.

Because of its mechanical strength, the implant will constitute a protective means for protecting both the regrowth activator and the bone substitute from the stresses induced by the reintroduction of loads onto the limb.

In essence, the wall of the implant can withstand the stresses, thus stabilizing the area in which the bone precursor is located.

The majority of the loads will be absorbed by the laterally fixed osteotomy plate, but this implant will contribute to stabilization.

The implant comprises an outer part 4 having a horseshoe shape, thus delimiting a hollow 5 in which a friable bone precursor 6 is housed.

The open end of this hollow 5 opens at the least thick area of the implant.

This horseshoe-shaped part 4 is delimited by a curved dorsal surface 4A and two lateral walls 4B whose outer surfaces 4C are flat.

Specifically because of the small portion of the osteotomy site that it occupies, this implant can be easily removed, particularly without weakening the bone regrowth.

The bone regrowth inside the hollow does not prevent the implant from being removed. In fact, the housing being open laterally, the implant can slide out past this bone formed at the level of the housing.

The front surfaces 4D of these lateral walls are flat and define a plane which ultimately contains said front surface 6A of the bone substitute 6 when it is put in place.

This implant 1 will be positioned so that the rear of the horseshoe-shaped part is located at the level of the cortex and the opening of the hollow is located near the center.

The inner lateral surfaces of the hollow are preferably parallel, as are the outer lateral surfaces, so as to facilitate the extraction of the implant after the regrowth of the bone.

The transverse dimension Z of the horseshoe-shaped wall is between 2 and 6 millimeters. It is on the order of the thickness of the cortex

This horseshoe-shaped part 4 will be made of a bioinert material having much better mechanical properties than that of the friable bone substitute 6.

This implant includes means 7 for maintaining a bone substitute inside the housing.

In order to maintain this substitute, the hollow of the horseshoe has, for example at the level of its base, a ledge 7A on which the inner bone substitute rests.

The base 8 of the inner bone substitute 6 is of reduced cross-section so as to be able to fit into the section delimited by the inner surface of the ledge 7A, for example with a height equal to the thickness of the aforementioned ledge.

The bone precursor is gently wedged into the hollow.

In a variant, at least one of the upper and lower surfaces of the horseshoe-shaped part 4 of the implant is fluted so as to limit sliding movements inside the bone.

The flutes will be limited to the area in which the spongy bone is located, i.e., to the branches of the hollow.

The rear part of the implant will not be fluted; this rear part is the part located at the level of the cortex. These flutes have an inclined orientation relative to a median vertical plane of symmetry. These flutes have a mirrored orientation, as may be seen in FIG. 5.

The advantage of this solution is that the outer part 4 of the implant provides strength in addition to that of the osteosynthesis plate and keeps the bone substitute from being stressed.

The outer part 4 has a protective effect because it absorbs stresses.

This implant will therefore transfer stresses from the bottom to the top and vice versa, just as the osteosynthesis plate must do, thus protecting the bone substitute from stresses so it can fulfill its function undisturbed.

At the back of the horseshoe-shaped part, a means 18 is provided for anchoring an ancillary device for the purpose of easily removing this implant.

For example, a threaded hole is provided.

The horseshoe shape facilitates the extraction of this implant by translation in a direction substantially perpendicular to the open side of the horseshoe.

Thus, it is understood that this implant is temporary and is intended to protect the inserted bone precursor during the time it takes for the bone to grow back. The presence of an osteotomy plate is necessary because this implant is not meant to support the entire weight of the body. The hollow shape makes it possible to remove it easily, and its small size prevents the bone reconstruction area from being weakened when this implant is removed.

1. Implant made of bioinert material to be used with an osteotomy plate to correct the alignment of the lower limbs by addition, said implant having a housing that opens onto its upper and lower surfaces, and being characterized in that it has sufficient mechanical strength to transmit and/or absorb loads to which it is subjected so as to protect a bone precursor placed inside the housing and is extractible without notable weakening of the bone reconstruction, and wherein the housing that houses the bone precursor has a hollow shape and the implant occupies a volume on the order of 10 to 35% of the osteotomy site.

2. Implant according to claim 1, characterized in that the mechanical properties of the implant are similar to those of the cortex of the bone.

3. Implant according to claim 1, characterized in that the material composing the implant is a polymer.

4. Implant according to claim 1, characterized in that it comprises means (7) for maintaining the bone precursor inside the housing.

5. Implant according to claim 4, characterized in that the hollow has, at the level of its base, a ledge (7A) on which the bone precursor rests.

6. Implant according to claim 5, characterized in that the base (8) of the bone precursor is of reduced cross-section so as to be able to fit into a section delimited by the inside surface of the ledge (7A).

7. Implant according to claim 1, characterized in that the hollow is delimited by a curved dorsal surface (4A) and two lateral walls (4B) whose outer surfaces (4C) are flat, thus forming a horseshoe.

8. Implant according to claim 1, characterized in that
the transverse dimension (Z) of horseshoe-shaped wall of the implant is between 2 and 6 millimeters.

9. Implant according to claim 1, characterized in that a means (18) are disposed at the back of a horseshoe-shaped part for anchoring an ancillary device for the purpose of extracting the implant.

10. Implant according to claim 3, characterized in that the polymer is polyetheretherketone, known by the acronym “PEEK”.

11. Implant according to claim 2, characterized in that the material composing the implant is a polymer.

12. Implant according to claim 2, characterized in that it comprises means (7) for maintaining the bone precursor inside the housing.

13. Implant according to claim 3, characterized in that it comprises means (7) for maintaining the bone precursor inside the housing.

14. Implant according to claim 12, characterized in that the hollow has, at the level of its base, a ledge (7A) on which the bone precursor rests.

15. Implant according to claim 13, characterized in that the hollow has at the level of its base, a ledge (7A) on which the bone precursor rests.

16. Implant according to claim 14, characterized in that the base (8) of the bone precursor is of reduced cross-section so as to be able to fit into the section delimited by the inside surface of the ledge (7A).

17. Implant according to claim 15, characterized in that the hollow has at the level of its base, a ledge (7A) on which the bone precursor rests.

18. Implant according to claim 11, characterized in that the polymer is polyetheretherketone, known by the acronym “PEEK”.

19. Implant according to claim 13, characterized in that the polymer is polyetheretherketone, known by the acronym “PEEK”.

20. Implant made of bioinert material to be used with an osteotomy plate to correct the alignment of the lower limbs by addition, said implant having a housing that opens onto its upper and lower surfaces, and being characterized in that it has mechanical properties similar to those of the cortex of the bone and sufficient mechanical strength to transmit and/or absorb loads to which it is subjected so as to protect a bone precursor placed inside the housing and is extractible without notable weakening of the bone reconstruction, and wherein the housing that houses the bone precursor has a hollow shape and the implant occupies a volume on the order of 10 to 35% of the osteotomy site, and includes means (7) for maintaining the bone precursor inside the housing and wherein the hollow shape is defined by walls forming a horseshoe shape having a transverse dimension between 2 and 6 millimeters.
